(19) United States
(12) Patent Application Publication
(10) Pub. No.: US 2009/0176888 A1
(43) Pub. Date: Jul. 9, 2009

(54) COMPOSITION COMPRISING PHYTOSPHINGOSINE OR DERIVATIVE THEREOF

(75) Inventors: Su Jae LEE, Seoul (KR); Yun Sil LEE, Seoul (KR); Soo Kwan KIM, Seoul (KR); Kyung Joong KIM, Seoul (KR); Chul Koo CHO, Seoul (KR); Chang Mo KANG, Seoul (KR); Tae Hwan KIM, Seoul (KR); Sangwoo BAE, Seoul (KR); Moon Taek PARK, Seoul (KR); Jung A. CHOI, Seoul (KR); Min Jeong KIM, Seoul (KR); Hee Yong CHUNG, Seongnam-city (KR); Sujong KIM, Suwon-city (KR); Seongnam KANG, Seoul (KR); Weon Ik CHOI, Seoul (KR); Jung A. KANG, Busan-city (KR)

Correspondence Address: CANTOR COLBURN, LLP, 20 Church Street, 22nd Floor, Hartford, CT, 06103, US

(73) Assignee: KOREA INSTITUTE OF RADIOLOGICAL & MEDICAL SCIENCES, Seoul (KR)

(21) Appl. No.: 12/399,384

(22) Filed: Mar. 6, 2009

Related U.S. Application Data
(62) Division of application No. 10/548,310, filed on Aug. 16, 2006.

(30) Foreign Application Priority Data
Mar. 7, 2003 (KR) PCT/KR2003/000445

(51) Int. Cl. A61K 31/13

(52) U.S. Cl. 514/669

(57) ABSTRACT

Provided is a composition for cancer treatment including phytosphingosine or a derivative thereof, or a pharmaceutically acceptable salt thereof as an active ingredient.

### CROSS-REFERENCE TO RELATED APPLICATIONS

This application is a divisional application of application Ser. No. 10/548,310 filed on Aug. 16, 2006, the entire contents of which are incorporated herein by reference.

### BACKGROUND

1. Field of the Invention

The present invention relates to a composition for cancer treatment or enhancement of radiosensitizing effect. More particularly, the present invention relates to a composition for cancer treatment or enhancement of radiosensitizing effect, which increases the sensitivity of cancer cells to radiation without side effects on normal cells.

2. Description of the Related Art

Anticancer therapy is largely classified into surgery, radiation, and chemotherapy. Alkylating agents, antibiotics, antimetabolites, plant derivatives, and steroids are used as anticancer chemotherapy drugs. Some drugs commonly used anticancer chemotherapy are Cisplatin as an alkylating agent, Doxorubicin hydrochloride as an antibiotic drug, Pentostatin as an antimetabolite drug, Taxol as a plant derivative drug, and Dexamethasone as a steroid drug. However, it is known that these anticancer drugs cause side effects such as damage to normal cells.

Presently, about 35% of Korean cancer patients and about 50% of American cancer patients undergo radiotherapy. The number of cancer patients who receive radiotherapy is increasing each year. Therefore, the importance of radiotherapy for cancer treatment is increasing.

Radiotherapy is necessary for treating various cancers. However, radiotherapy has problems such as cellular resistance to radiation and damage to normal cells due to a high dose of radiation, thereby decreasing radiotherapy efficiency.

Therefore, considerable efforts have been made to develop radiosensitizers for increasing the radiotherapy efficiency. In this regard, attempts have been made to increase radiosensitivity in several solid tumors such as breast cancer, uterine cervical cancer, lung cancer, gastric cancer, and large intestine cancer (or colorectal cancer) using Taxol and Cisplatin that are presently known as anticancer agents. It was reported that as a result of administration of Taxol or Cisplatin in combination with radiotherapy in solid tumor patients, the radiotherapy efficiency was enhanced [Amorino et al., “Enhancement of Radiation Effects by Combined Decetaxel and Carboplatin Treatment in vitro”, Radiat Oncol Investig 1999; 7(6): 343-352; Choy H., “Taxanes in Combined-Modality Therapy for Solid Tumor”, Oncology, 1999 October; 13:22-38; Safran H et al., “Paclitaxel, Cisplatin, and Concurrent Radiation for Esophageal Cancer”, Cancer Invest 2001; 19(1): 1-7]. However, these anticancer agents have a serious side effect and can be applied only to specific cancer cells.

### SUMMARY OF THE INVENTION

The present invention provides a composition for cancer treatment or enhancement of radiosensitizing effect, which has a treatment or enhancement effect on various cancer cells without side effects on normal cells.

According to an aspect of the present invention, there is provided a composition for cancer treatment comprising a compound represented by formula 1 or a pharmaceutically acceptable salt thereof:

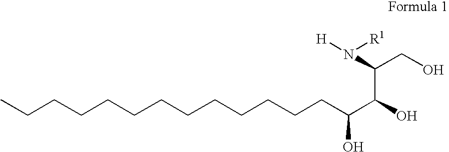

wherein, R¹ is hydrogen or a substituted or unsubstituted C₁-C₂₀ alkylcarbonyl group.

According to another aspect of the present invention, there is provided a composition for enhancement of radiosensitizing effect comprising a compound of formula 1 or a pharmaceutically acceptable salt thereof. The composition has a radioenhancement effect on various cancer cells without side effects on normal cells.

### BRIEF DESCRIPTION OF THE DRAWINGS

The above and other features and advantages of the present invention will become more apparent by describing in detail exemplary embodiments thereof with reference to the attached drawings in which:

FIG. 1 is a graph showing an anticancer effect of phytosphingosine on various human cancer cells;

FIG. 2 is a graph showing anticancer effects of various phytosphingosine derivatives on human lung cancer cells;

FIG. 3 is a graph showing an anticancer effect of phytosphingosine on human uterine cervical cancer cells;

FIG. 4 is a graph showing an anticancer effect of phytosphingosine on human breast cancer cells;

FIG. 5 is a graph showing an anticancer effect of phytosphingosine on human lung cancer cells;

FIG. 6 is a graph showing an anticancer effect of phytosphingosine on human blood cancer cells;

FIG. 7 shows changes in mitochondrial membrane potential and cytochrome c as a function of time of exposure to phytosphingosine in human lung cancer cells using flow cytometry;

FIG. 8 is a graph showing reduction in mitochondrial membrane potential as a function of time of exposure to phytosphingosine in human lung cancer cells;

FIG. 9 is a photograph showing increase in cytochrome c as a function of time of exposure to phytosphingosine;

FIG. 10 is graphs showing increase in caspase activity as a function of time of exposure to phytosphingosine in human lung cancer and blood cancer cells;

FIG. 11 is a graph showing an anticancer effect of phytosphingosine on nude mice transplanted with human uterine cervical cancer cells;

FIG. 12 is graphs showing changes in the sensitizer enhancement ratio (SER) of sphingosine, phytosphingosine, and their derivatives in human lung cancer cells;

FIG. 13 is a graph showing enhancement of radiosensitizing effect on human lung cancer cells by phytosphingosine or a derivative thereof;

FIG. 14A is a graph showing enhancement of radiosensitizing effect by concurrent application of C8PS and radiation when compared to radiation alone at LD₅₀ of human lung cancer cells; and FIG. 14B is a graph showing different enhancement of radiosensitizing effects of Taxol and C8PS at LD₅₀ of human lung cancer cells;

FIG. 15 is a graph showing enhancement of radiosensitizing effect on human blood cancer cells by phytosphingosine or a derivative thereof;

FIG. 16 is a graph showing enhancement of radiosensitizing effect on human blood cancer cells by phytosphingosine or a derivative thereof as a function of time;

FIG. 17 is a graph showing enhancement of radiosensitizing effect on human uterine cervical cancer and breast cancer cells by phytosphingosine or a derivative thereof;

FIG. 18 is graphs showing changes in the SER of C6PS in human uterine cervical cancer and breast cancer cells;

FIG. 19 is a photograph showing enhancement of radiosensitizing effect on human lung cancer cells by C8PS as revealed by DAPI staining;

FIG. 20 is a photograph showing enhancement of radiosensitizing effect on human lung cancer cells by C8PS as analyzed by DNA fragmentation;

FIG. 21 is a graph showing enhancement of human lung cancer cell apoptotic rate by concurrent application of C8PS and radiation as revealed by DAPI staining;

FIG. 22 is photographs showing change in tumor size after administration of C8PS in nude mice transplanted with human lung cancer cells;

FIG. 23 is a graph showing change in tumor size as a function of days after administration of C8PS in nude mice transplanted with human lung cancer cells; and

FIGS. 24 and 25 are graphs showing changes in tumor size as a function of days after administration of phytosphingosine derivatives, C4PS and C6PS, respectively, in nude mice transplanted with human uterine cervical cancer cells.

### DETAILED DESCRIPTION OF THE INVENTION

Hereinafter, the present invention will be described in more detail.

The term, “radiosensitizer” as used herein means a substance that is administered in combination with radiotherapy for increasing sensitivity of cancer cells to radiation. Therefore, radiotherapy efficiency for killing cancer cells or inhibiting growth of cancer cells is increased.

The present invention provides a composition for cancer treatment or for enhancement of radiosensitizing effect comprising a compound of formula 1 or a pharmaceutically acceptable salt thereof:

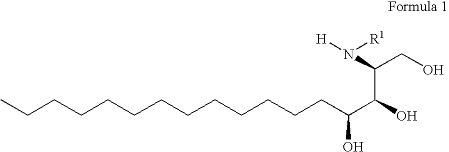

wherein, R¹ is hydrogen or a substituted or unsubstituted C₁-C₂₀ alkylcarbonyl group.

The present inventors found that after administration of phytosphingosine or a derivative thereof of formula 1 to various cancer cells for anticancer treatment, apoptotic cell death of cancer cells was promoted.

Phytosphingoshine as used in the cancer treatment composition of the present invention is a plant-derived, cell membrane lipid metabolite. The precise physiological metabolism and the function of phytosphingoshine as an anticancer agent are not yet known.

There are no particular limitations to a phytosphingosine derivative to be used in the composition of the present invention provided that it has a fundamental structure of phytosphingosine. Preferable phytosphingosine derivatives are those that R¹ is hydrogen, ethanoyl group, propanoyl group, butanoyl group, pentanoyl group, hexanoyl group, heptanoyl group, octanoyl group, nonanoyl group, decanoyl group, undecanoyl group, or dodecanoyl group. More preferably, R¹ is hydrogen, butanoyl group, hexanoyl group, or octanoyl group.

The phytosphingosine derivative in which R¹ is an alkylcarbonyl group can be easily introduced in cancer cells while maintaining structural stability. Like phytosphingosine, there were no reports about the precise physiological metabolism and the function as an anticancer agent of the phytosphingosine derivative.

The phytosphingosine derivative can be easily obtained by acylation of an amino group on phytosphingosine. In this case, acylation can be induced by using acid, anhydride, ester, or amide. Alternatively, phytosphingosine derivative is commercially available (Doosan Biotech, Korea).

The composition of the present invention comprises a compound of formula 1 or a pharmaceutically acceptable salt thereof. There are no particular limitations to the salt provided that is pharmaceutically acceptable. Examples of the salt comprise an acid addition salt of hydrochloric acid, sulfuric acid, nitric acid, phosphoric acid, hydrofluoric acid, hydrobromic acid, formic acid, acetic acid, tartaric acid, lactic acid, citric acid, fumaric acid, maleic acid, succinic acid, methanesulfonic acid, benzenesulfonic acid, and naphthalenesulfonic acid.

The composition of the present invention may comprise a pharmaceutically acceptable carrier. The pharmaceutically acceptable carrier to be used in the present invention may comprise excipient, disintegrator, binding agent, lubricant, and other additivies such as stabilizer, palliative, and emulsifier. Examples of the excipient comprise microcrystal cellulose, lactose, and lower substituted hydroxycellulose and examples of the disintegrator comprise sodium starch glycolate and anhydrous calcium mono-hydrogen phosphate. Examples of the binding agent comprise polyvinylpyrrolidone, lower substituted hydroxypropylcellulose, and hydroxypropylcellulose and examples of the lubricant comprise magnesium stearinate, silicon dioxide, and talc.

The composition of the present invention may be formulated in a form of granule, powder, liquid, tablet, capsule, or dry syrup for oral administration or in a form of injection for parenteral administration. Preferably, the composition of the present invention is orally administered in a form of a liquid preparation which is dissolved in ethanol.

According to the present invention, a therapeutically effective amount of a compound of formula 1 or a pharmaceutically acceptable salt thereof for cancer treatment or enhancement of radiosensitizing effect may be 50 to 2,000 mg/kg/day. However, the therapeutically effective amount and the unit dosage form can vary depending on radiation dose, age, sex, and condition of a patient.

Meanwhile, phytosphingosine analogue, sphingosine was reported to be involved in growth, differentiation, and death of cells and induces apoptosis in liver cancer cells. However, the precise physiological mechanism of sphingosine are not yet known.

In the present invention, anticancer effects of phytosphingosine and a derivative thereof were demonstrated both in vitro and in vivo experiments.

According to the experiment results, phytosphingosine and a derivative thereof of formula 1 induced apoptosis of uterine cervical cancer, breast cancer, and lung cancer cells. The apoptotic effects of phytosphingosine and a derivative thereof on various cancer cells exhibited a concentration- and post-treatment time-dependent increase. For example, 15 μg/ml of phytosphingosine induced 50% apoptosis at 12-18 hours after treatment to human uterine cervical cancer cells, 10 μg/ml of phytosphingosine induced 50% apoptosis at 12 hours after treatment to human breast cancer cells, and 10 μg/ml and 5 μg/ml of phytosphingosine induced 50% or more apoptosis at 3-6 hours after treatment to human lung cancer and blood cancer cells, respectively.

In the present invention, experiments showing a relationship between mitochondrial membrane potential and cytochrome c release were carried out. According to the experiment results in cultured lung cancer cells, when cancer cells were treated with phytosphingosine, the mitochondrial membrane potential of cancer cells was decreased. As a result, the release of cytochrome c as an apoptosis-related factor from mitochondria was increased. Therefore, the activity of caspase as an apoptosis factor which is directly involved in the induction of apoptosis was considerably increased, thereby increasing incidence of apoptosis. Without being limited to any particular theory, it is presumed from these facts that the induction of apoptosis by phytosphingosine is caused by caspase, which is activated when cytochrome c is released due to the reduction of mitochondrial membrane potential.

Phytosphingosine or a derivative thereof also exhibited an anticancer effect in an animal test. In this case, cancer cell transplanted nude mice were used as animal models. In detail, phytosphingosine was orally administered to nude mice transplanted with human uterine cervical cancer cells at dosages of 50 mg/kg/day for one week. Then, tumor size was daily measured for a period of 40 days after phytosphingoshine treatment. According to the experiment results, unlike the untreated nude mice as a control group, tumor size in a phytosphingosine-treated group did not show changes for 20 days. Even at the 40th day, tumor growth was observed but the degree of the growth was slight. Consequently, the phytosphingosine-treated group exhibited the potent inhibitory effect on tumor growth, when compared to the control group.

According to oral or transdermal toxicity tests performed on rats, phytosphingosine or a derivative thereof exhibited LD₅₀ (the concentration which induces 50% of cell death) of 2000 mg/kg or more. As a result of the tests, it was demonstrated that phytosphingosine or a derivative thereof exhibits little side effects while maintaining high physiological safety.

From the above test results, it can be seen that phytosphingosine and a derivative thereof exhibit an anticancer effect on human lung cancer cells, uterine cervical cancer cells, breast cancer cells, and blood cancer cells without side effects.

Phytosphingosine or a derivative thereof was administered to various cancer cells in combination with radiotherapy. As a result, the apoptotic rate of cancer cells was increased, when compared to radiation alone treated cancer cells. Therefore, it can be seen that the administration of phytosphingosine or a derivative thereof causes to increase in radiotherapy efficiency.

Up until now, there were no reports that phytosphingoshine and a derivative thereof served as radiosensitizers.

The present inventors demonstrated an enhancement of radiosensitizing effect of phytosphingosine and a derivative thereof through both in vitro and in vivo experiments.

According to one embodiment of the present invention, cancer cells which mainly rely on radiotherapy, uterine cervical cancer cells, breast cancer cells, and lung cancer cells were treated with phytosphingosine or a derivative thereof in combination with radiation. As a result, in the case of all the above cancer cells, the apoptotic rate of cancer cells was remarkably increased by 30% or more, when compared to radiation alone treated cells. In addition, in animal tests using cancer cell transplanted nude mice, the concurrent application of radiation and phytosphingosine or a derivative thereof resulted in a further reduction of tumor growth than radiation alone treatment.

From the above test results, it can be seen that phytosphingosine or a derivative thereof significantly enhances the radiotherapy efficiency on human lung cancer cells, uterine cervical cancer cells, breast cancer cells, and blood cancer cells without side effects. Therefore, phytosphingosine and a derivative thereof can be effective as active ingredients for radiosensitizers.

Hereinafter, the present invention will be described more specifically by examples. However, the following examples are provided only for illustrations and thus the present invention is not limited to or by them.

The following abbreviations are used for subjects shown against each throughout the specification and drawings.

Cont: control group, IR: radiation, PS: phytosphingosine, C4PS: N-butanoyl phytosphingosine, C6PS: N-hexanoyl phytosphingosine, C8PS: N-octanoyl phytosphingosine, and C12PS: N-dodecanoyl phytosphingosine.

### Experiment 1

Cancer cells were treated with phytosphingosine, C4PS, C6PS, C8PS, and C12PS (Cosmoferm, Germany) and anticancer effects were evaluated in the following manners.

Cancer cells as used in the experiment were human lung cancer cells (NCI-H460, Korean Cell Line Bank), human breast cancer cells (MDA-MB-231, American Type Culture Collection (ATCC)), human uterine cervical cancer cells (HeLa, Korean Cell Line Bank), and blood cancer cells (Jurkat, ATCC). These cancer cells were cultured in RPMI 1640 media containing 10% FBS, penicillin, and streptomycin (GIBCO BRL).

### 1. Apoptosis Test

The apoptosis tests were carried out at human lung cancer cells, breast cancer cells, uterine cervical cancer cells, and blood cancer cells as follows:

Phytosphingosine or a derivative thereof was dissolved in DMSO. Samples of the resultant solution of different concentration levels (1, 2, 5, 10, 15, 20 μg/ml) were prepared. According to the following treatment schedule, phytosphingosine treated cancer cells were cultured, washed with PBS (phosphate buffered saline), and fixed with 70% ethanol. The fixed cells were again washed with PBS, suspended in PBS, and 1 mg/ml of RNase was added thereto. Then, DNA was stained with 50 μg/ml of propidium iodide fluorescent dye and the change in the Sub G1 was analyzed by flow cytometry (Becton DICKINSON). The Sub G1, a marker of apoptotic cell death, means DNA distribution lower than that in the G1 phase of cell cycle.

Phytosphingosine Treatment Schedule:

(1-1) In order to determine an anticancer effect of phytosphingosine, 5 ml of 10 μg/ml of phytosphingosine was added to respective dishes containing lung cancer cells, breast cancer cells, uterine cervical cancer cells, and blood cancer cells.

(1-2) In order to examine a correlation of an anticancer effect with the phytosphingosine concentration and post-treatment culture time, each 5 ml of 2, 5, 10, and 15 μg/ml of phytosphingosine was added to respective human uterine cervical cancer cell-containing dishes and human breast cancer cell-containing dishes. In addition, each 5 ml of 5, 10, 15, and 20 μg/ml of phytosphingosine was added to respective human lung cancer cell-containing dishes, and each 5 ml of 1, 5, 10, and 20 μg/ml of phytosphingosine was added to respective human blood cancer cell-containing dishes.

(1-3) An anticancer effect of phytosphingosine derivatives was determined in the same manner as mentioned in (1-1) and (1-2) except using 5 μg/ml of C4PS, C6PS, C8PS, and C12PS.

### Test Result

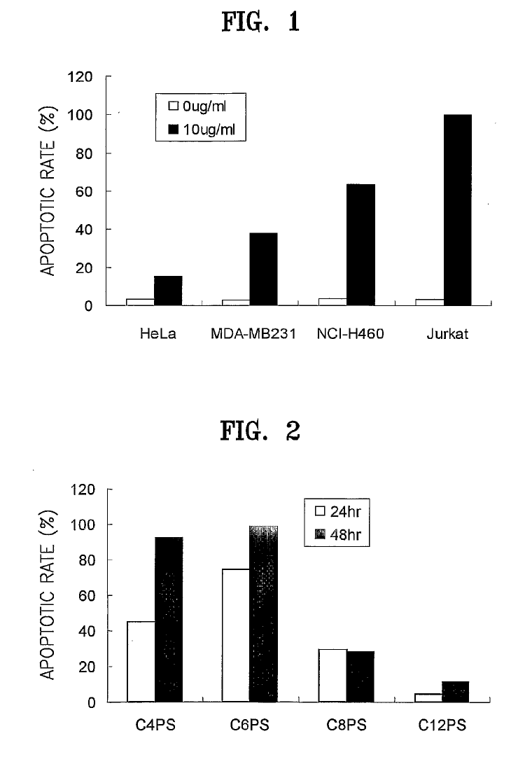

The test (1-1) results of anticancer effects of phytosphingosine on human cancer cells are shown in FIG. 1. As shown in FIG. 1, phytosphingosine-treated cancer cells exhibited excellent anticancer effects, when compared to the untreated-cancer cells. In particular, the apoptotic rate in lung cancer and blood cancer cells was significantly increased to 50% or more.

The test (1-3) results of anticancer effects of phytosphingosine derivatives, C4PS, C6PS, C8PS, and C12PS on human lung cancer cells are shown in FIG. 2. As shown in FIG. 2, all the phytosphingosine derivatives exhibited anticancer effects. In particular, in case of C4PS and C6PS, the apoptotic rates at the culture time of 48 hours reached almost 100%.

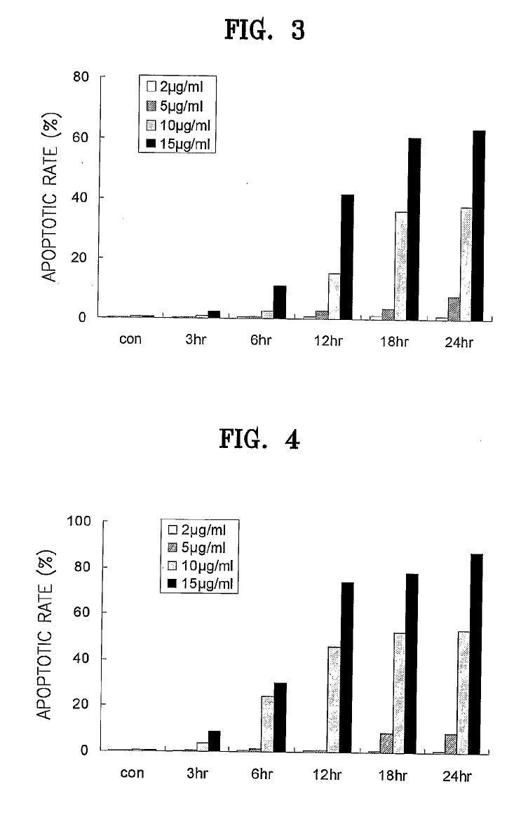

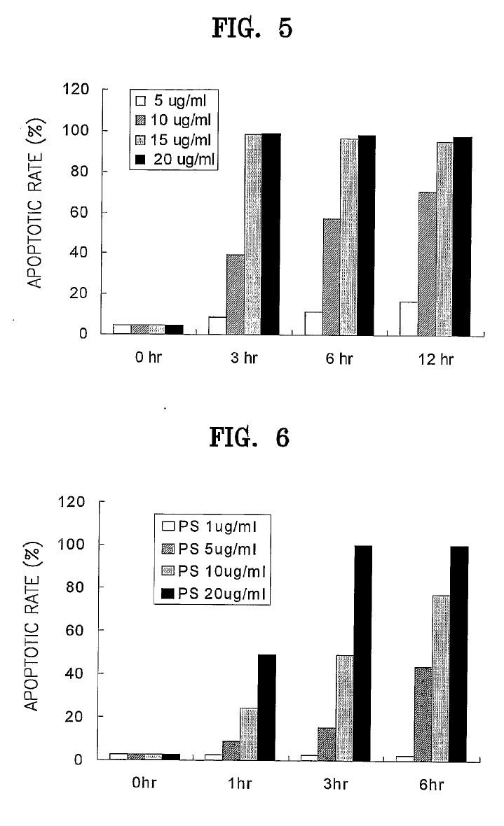

The test (1-2) results of a correlation of an anticancer effect with the phytosphingosine concentration and post-treatment culture time are shown FIGS. 3, 4, 5, and 6. 15 μg/ml or more of phytosphingosine induced 50% or more apoptosis at culture of 12 hours or more after treatment to human uterine cervical cancer cells (see FIG. 3), 10 μg/ml or more of phytosphingosine induced 50% or more apoptosis at culture of 12 hours or more after treatment to human breast cancer cells (see FIG. 4), 10 μg/ml or more of phytosphingosine induced 50% apoptosis at culture of 6 hours or more after treatment to human lung cancer cells (see FIG. 5), and 5 μg/ml or more and 10 μg/ml or more of phytosphingosine induced 50% or more apoptosis at culture of 6 hours or more and 3 hours or more after treatment to human blood cancer cells, respectively (see FIG. 6).

As apparent from the above, the anticancer effect of phytosphingosine of the present invention is proportional to phytosphingosine concentration and post-treatment culture time. In particular, the anticancer effect of phytosphingosine on lung cancer cells and blood cancer cells was excellent.

### 2. Analysis of Mitochondrial Membrane Potential and Western Blotting

Human lung cancer cells and blood cancer cells were cultured under the same condition as in the apoptosis test and were treated with 10 ml of phytosphingosine (10 μg/ml of phytosphingosine for lung cancer cells, and 5 μg/ml and 10 μg/ml of phytosphingosine for blood cancer cells). Then, the analysis of mitochondrial membrane potential and western blotting was carried out.

### (2-1) Analysis of Mitochondrial Membrane Potential

Mitochondria were stained with 30 nM of a specific DioC6(3) dye (Calbiochem) for 30 minutes and culture media were removed. Then, lung cancer cells were twice washed with PBS and the membrane potential was analyzed by flow cytometry.

### (2-2) Western Blotting

Phytosphingosine-treated human lung cancer cells and blood cancer cells were dissolved in a protease inhibitor-containing lysis buffer (40 mM Tris-Cl (pH 8.0), 120 mM NaCl, 0.1% Nonidet-P4) and centrifuged to give a protein extract. Pure proteins were isolated from the protein extract using SDS-PAGE and transferred to a nitrocellulose membrane. The protein-bound nitrocellulose membrane was blocked with skim milk and incubated with as caspase-3, caspase-8, caspase-9, and poly(ADP-ribose)polymerase (PARP) as primary antibodies at room temperature for one hour. The primary antibodies-bound nitrocellulose membranes were three times washed with PBS-T (phosphate buffered saline, 0.1% Tween-20) and incubated with HRP (Horse Radish Peroxidase)-conjugated secondary antibodies for one hour. The expression of caspase-3, caspase-8, caspase-9, and PARP was detected using ECL reagent (PerkinElmer Life Science, Inc.).

### Test Result

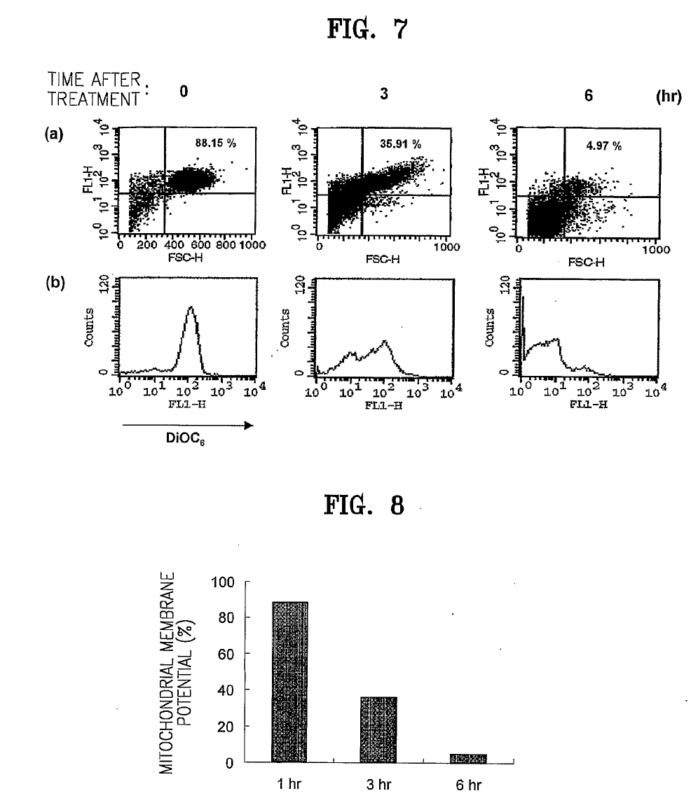

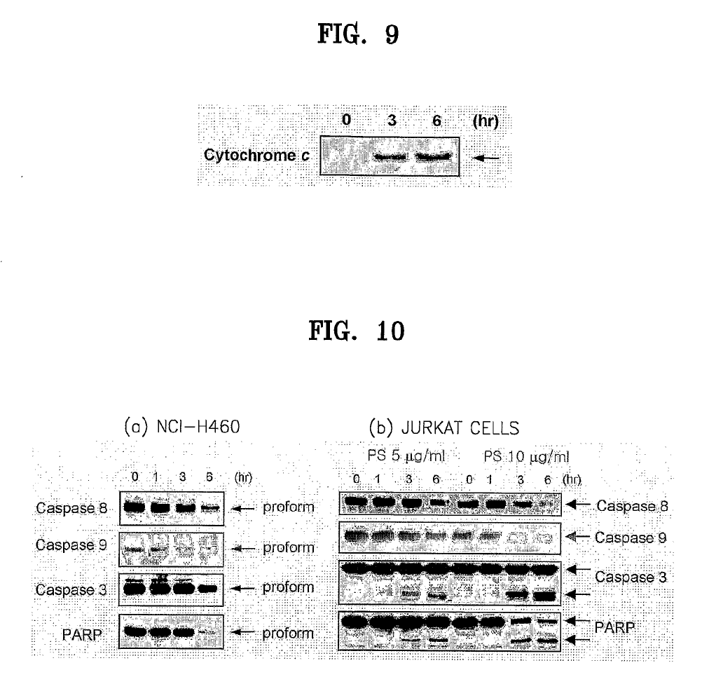

When lung cancer and blood cancer cells were treated with phytosphingosine, the mitochondrial membrane potential of these cancer cells was decreased. As a result, the release of cytochrome c as an apoptosis-related factor from mitochondria was increased (see FIGS. 7-9). Therefore, the activities of the caspases as apoptosis factors which are directly involved in the induction of apoptosis were considerably increased, thereby increasing the incidence of apoptosis (see FIG. 10). It is presumed from these facts that the induction of apoptosis and the inhibition of tumor growth by phytosphingosine are caused by caspase, which is activated when cytochrome c is released due to the reduction of mitochondrial membrane potential.

### 3. In Vivo Animal Test

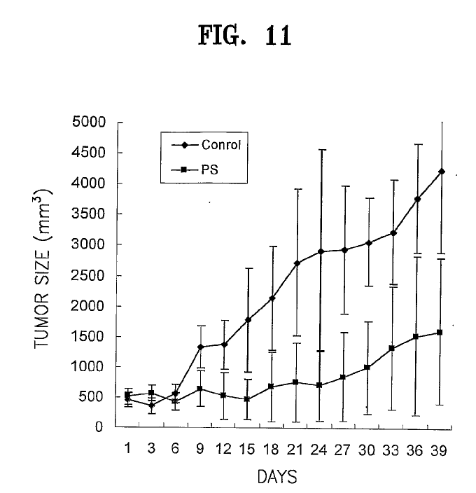

Nude mice (body weight: about 20 g) were randomized into 2 groups: a first group is for a control group and a second group is for treatment with phytosphingosine. The femoral region of the nude mice was transplanted with human uterine cervical cancer cells (NCI-H460 cells). Then, tumor volume was allowed to reach a level of 120-150 cm³. A 50 mg/kg solution of phytosphingosine in an olive oil was orally administered to the second group for one week on a daily basis. Tumor volume was measured at intervals of 3 days for 40 days and the results are presented in FIG. 11.

According to the experiment results, unlike the control group, tumor size in the phytosphingosine-treated group did not show changes for 20 days. Even at the 40th day, the phytosphingosine-treated group exhibited the potent inhibitory effect on tumor growth, when compared to the control group.

### Experiment 2

In order to demonstrate the enhancement of radiosensitizing effect of phytosphingosine, C4PS, C6PS, C8PS, and C12PS, cancer cells were treated with these drugs in combination with radiation. Specific experimental methods are as follows.

Experimental materials were prepared in the same manner as in Experiment 1.

### 1. Colony Formation Test and Apoptosis Test

### (1-1) Colony Formation Test

Colony formation tests were carried out in human lung cancer cells, breast cancer cells, and uterine cervical cancer cells as follows:

Respective sphingosine, phytosphingosine, C6PS, and C8PS were dissolved ethanol to produce specimens. About 600 cells (for each cancer) were plated in a dish with a diameter of 60 mm and incubated in a CO₂ incubator at 37° C. for a day. Then, the cancer cells were treated with the specimens in combination with radiation and were continuously cultured. When suitable colonies were formed, the cancer cells were fixed with a fixing solution (methanol/acetic acid=3:1) and stained with trypan blue. Then, the number of the colonies was counted and the results were evaluated in a comparative manner.

### (1-2) Apoptosis Test

(1) Each 5 ml of 20 μg/ml of phytosphingosine and derivatives thereof were added to human lung cancer cell-containing dishes. Some cells were cultured without radiation and others were cultured with radiation with dose of 4 Gy. This apoptosis test was carried out in the same manner in the Experiment 1. Specimens used in this apoptosis test are as follows:

Control (Cont), IR, PS, PS+IR, C4PS, C4PS+IR, C6PS, C6PS+IR, C8PS, C8PS+IR, C12PS, C12PS+IR.

(2) Apoptosis tests for human blood cancer cells, uterine cervical cancer cells, and breast cancer cells were carried out in the same manner as in the above (1) except using 5 μg/ml of phytosphingosine and derivatives thereof.

### Test Result

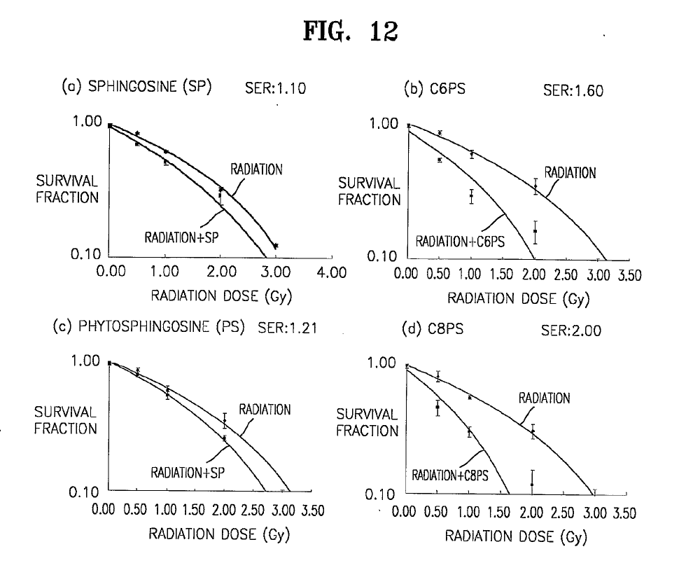

Through the aforementioned colony formation test and apoptosis test, the enhancement of radiosensitizing effect of phytosphingosine or derivatives thereof on human lung cancer cells, blood cancer cells, uterine cervical cancer cells, and breast cancer cells was examined. As shown in FIG. 12, the inhibitory effect on tumor growth in a phytosphingosine (or a derivative thereof) and radiation concurrent treated group was increased, when compared to a phytosphingosine (or a derivative thereof) or radiation alone treated group.

According to the colony formation test in human lung cancer cells, the tumor growth in a PS and radiation concurrent treated group was reduced by 30% or more, when compared to a radiation, sphingosine, or PS alone treated group (FIG. 12).

According to the test results of radiosensitivity of human lung cancer cells by phytosphingosine or derivatives thereof, C8PS exhibited excellent radiosensitivity to human lung cancer cells. As shown in FIG. 12, C8PS exhibited the sensitizer enhancement ratio (SER) of 1.10 for sphingosine, 1.21 for phytosphingosine, 1.6 for C6PS, and 2 for C8PS. Therefore, all the phytosphingosine and derivatives thereof exhibited the enhancement of radiosensitizing effect.

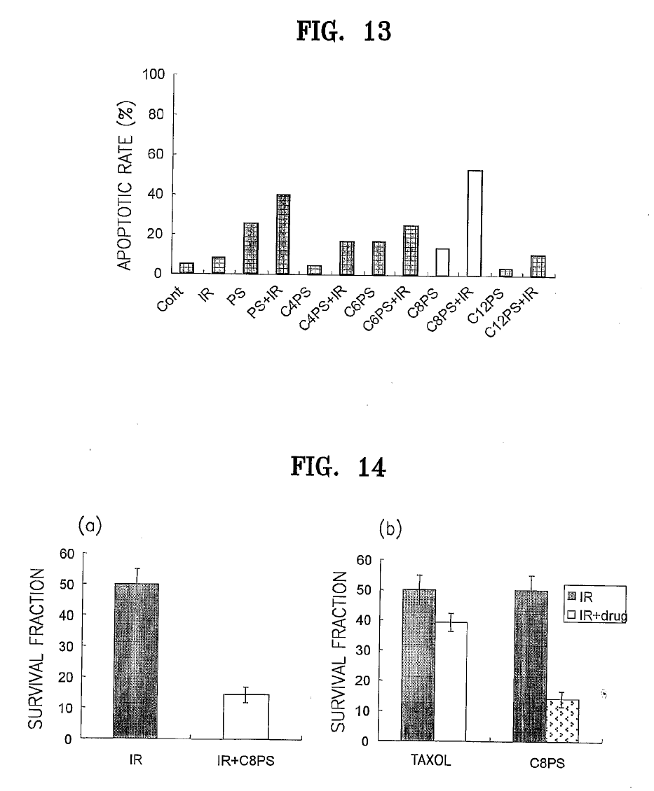

According to the apoptotic results of human lung cancer cells when phytosphingosine or derivatives thereof was applied in combination with radiation, it was demonstrated that the concurrent application of C8PS and radiation exhibited an excellent apoptotic effect (FIG. 13). With reference to the number of colonies, the number of colonies was reduced to 50% in radiation alone-treatment. On the other hand, when C8PS was applied in combination with radiation, the apoptotic rate of human lung cancer cells was increased by about 30%, when compared to radiation alone treatment (FIG. 14A).

Radiosensitivities of Taxol as a well known radiosensitizer and C8PS to human lung cancer cells were examined and the results are presented in FIG. 14B. As shown in FIG. 14B, the radiosensitivity of C8PS was increased by 20% or more relative to Taxol.

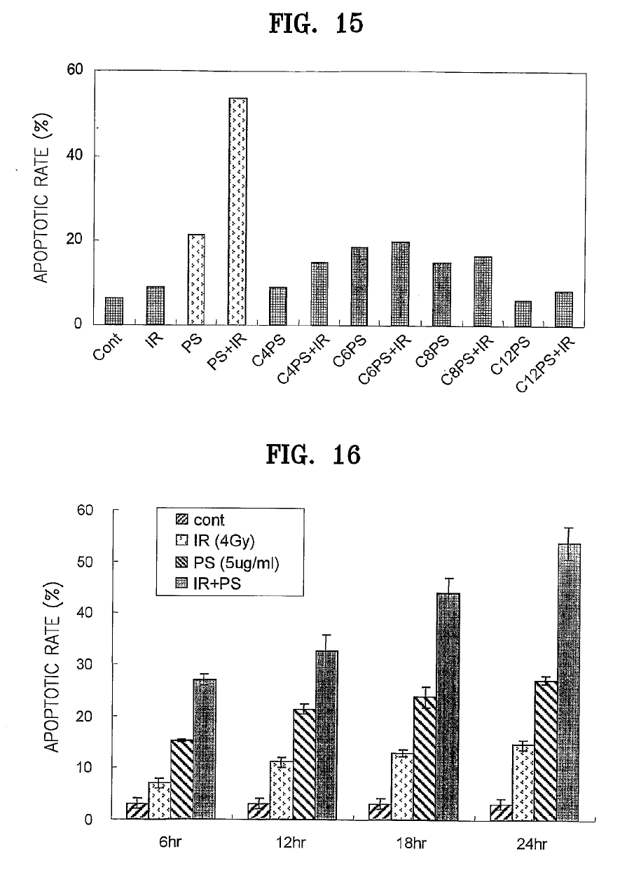

The enhancement of radiosensitizing effect on human blood cancer cells by phytosphingosine or derivative thereof was examined. As a result, it was demonstrated that phytosphingosine and derivatives have radiosensitizing effects on human blood cancer cells. In particular, the apoptotic rate in the PS and radiation concurrent treated group was increased by about 20% or more, when compared to the PS or radiation alone treated group (FIG. 15). The enhancement of radiosensitizing effects of phytosphingosine and derivatives thereof as a function of time was tested in human blood cancer cells. The apoptosis in the PS and radiation concurrent treated group occurred in a time-dependent increase manner. After 18 hours, the apoptotic rate in the PS and radiation concurrent treated group was increased by about 15% or more, when compared to the PS or radiation alone treated group (FIG. 16).

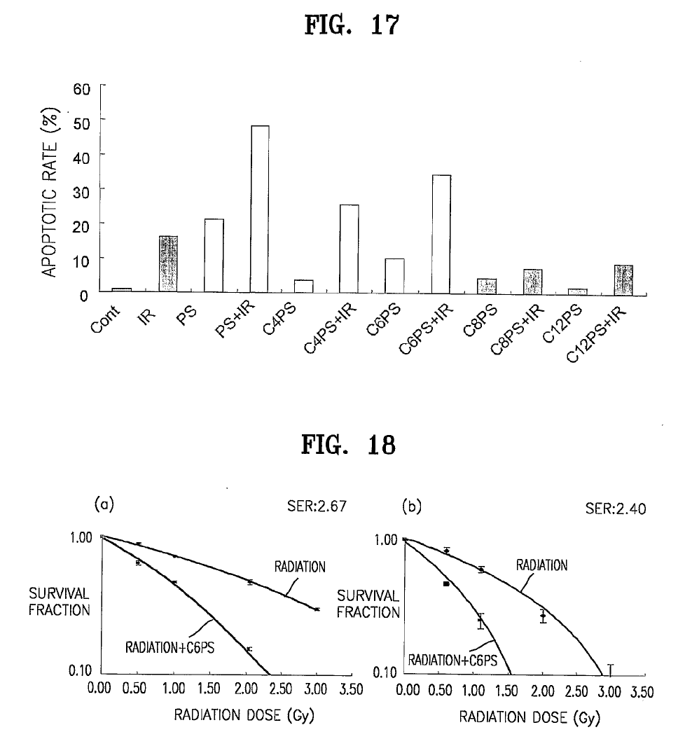

The enhancement of radiosensitizing effect on human uterine cervical cancer cells and breast cancer cells by phytosphingosine and derivatives thereof were examined. As a result, the enhancement of radiosensitizing effects of PS, C4PS, and C6PS were excellent (FIG. 17). The radiosensitivities of C6PS to human uterine cervical cancer cells and breast cancer cells were analyzed through the colony formation test and the results are presented in FIG. 18. As shown in FIG. 18, C6PS exhibited the SER of 2.67 for human uterine cervical cancer cells and 2.40 for human breast cancer cells.

### 2. DAPI Staining and DNA Fragmentation

Human lung cancer cells were cultured in the same manner as in Experiment 1 and injected with 5 ml of 20 μg/ml of C8PS. Then, the DAPI staining and DNA fragmentation were carried out.

### (2-1) DAPI Staining and DNA Fragmentation

DAPI staining protocol was as follows:

First, a control cell group, a radiation-treated cell group, a C8PS-treated cell group, and a C8PS and radiation concurrent treated cell group were fixed with 4% paraformaldehyde at room temperature for 30 minutes and washed with PBS. 50 ng/ml of a DAPI solution was added to the fixed cell groups and incubated for 30 minutes. Then, the cell groups were again washed with PBS and examined with a fluorescent microscope. Cellular apoptosis is characterized by condensation and fragmentation of cell nuclei. Based on this fact, apoptotic cells were counted in each group. Then, the number of apoptotic cells was divided by the number of total cells to derive the percentage of apoptotic cells in each group.

DNA fragmentation was carried out as follows:

C8PS-treated human lung cancer cells were dissolved in a lysis buffer (20 mM Tris/HCl, pH 8.0, 0.1 mM EDTA, 1% SDS and 0.5 mg/ml proteinase K) and treated with a mixture of phenol, chloroform, and isoamylalcohol (phenol/chloroform/isoamylalcohol=25:24:1) to thereby give a chromosomal DNA extract. The chromosomal DNA extract was subjected to 1% agarose gel electrophoresis and the resulting DNA fragments were visualized under an UV light.

### Test Result

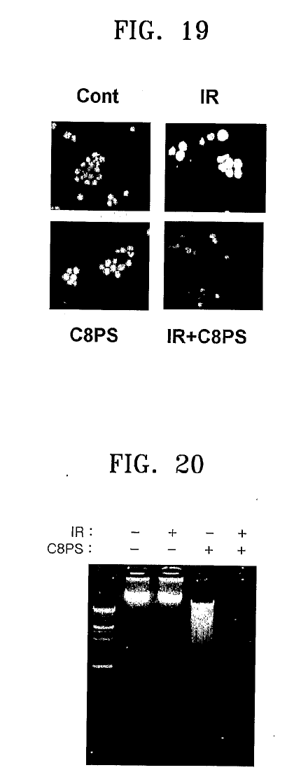

DAPI staining and DNA fragmentation were performed to demonstrate how C8PS increases the radiosensitizing effect on human lung cancer cells. According to the result of DAPI staining as shown in FIG. 19, the radiation and C8PS concurrent treated group exhibited higher apoptotic rate than the radiation or C8PS alone treated group. Similarly, chromosomal DNA fragmentation was remarkably increased in the radiation and C8PS concurrent treated group, when compared to the radiation or C8PS alone treated group (see FIG. 20). These results suggest that the radiosensitizing effect is increased by C8PS-mediated apoptosis.

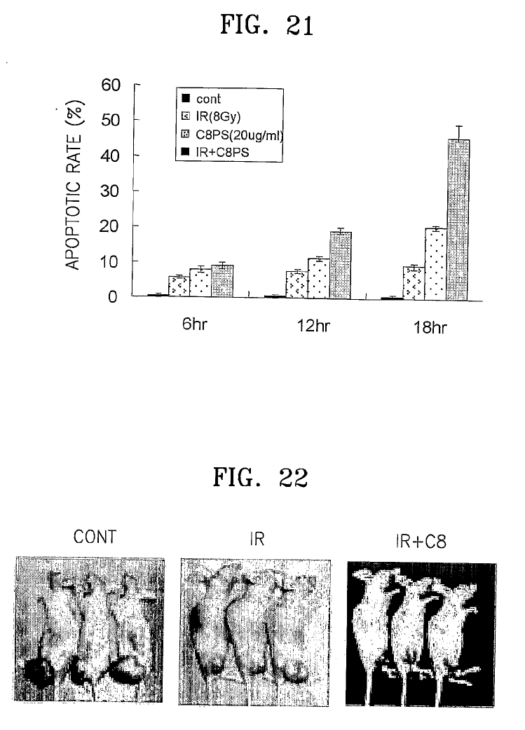

In addition, DAPI staining demonstrated that C8PS increases the radiosensitizing effect on human lung cancer cells as a function of time. In detail, the radiation and C8PS concurrent treated group exhibited higher apoptotic rate in a time-dependent manner, when compared to the radiation or C8PS alone treated group. In particular, the apoptotic rate in the radiation and C8PS concurrent treated group was increased by 20% or more, when compared to the radiation or C8PS alone treated group (FIG. 21).

### 3. In Vivo Animal Test

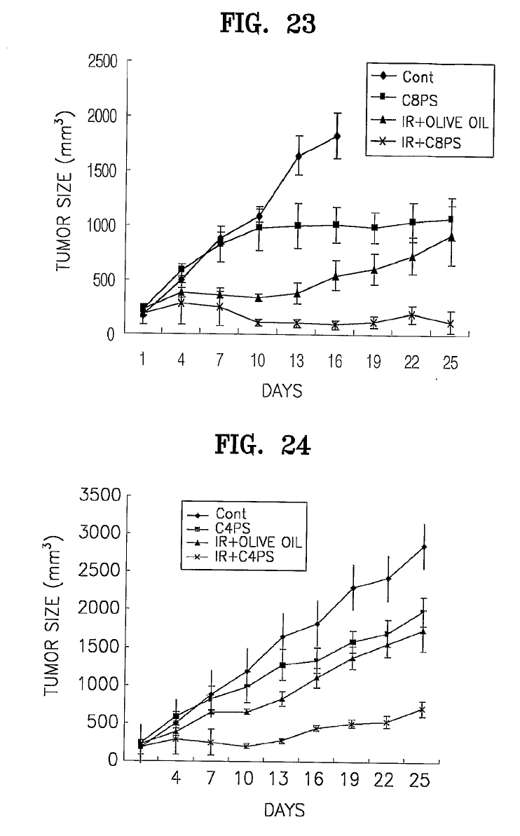

Nude mice (body weight: about 20 g) were randomized into 4 groups and the femoral region of the nude mice transplanted with human lung cancer cells. Then, tumor volume was allowed to reach a level of 120-150 cm³. One group had untreated cells as a control and a second group had radiation (dose of 20 Gy)-treated cells. A third group was orally administered with 50 mg/kg of an olive oil for one week on a daily basis, followed by radiotherapy (dose of 20 Gy). A fourth group was orally administered with a 50 mg/kg solution of phytosphingosine in an olive oil for one week on a daily basis, followed by radiotherapy (dose of 20 Gy). Tumor volume was measured at intervals of 3 days for 40 days. As a result, the tumor volume of the radiation and C8PS concurrent treated group was significantly reduced, when compared to the radiation or C8PS alone treated group (FIG. 22). With reference to correlation between a tumor size and a culture day in human lung cancer cells, the tumor size of a control group rapidly increased in a culture in a day-dependent manner. In case of a C8PS alone treated group, the tumor size increased until 10 days after the treatment. However, after the 10th day, the tumor size showed little changed. The tumor size in a radiation alone treated group slowly increased in a culture in a day-dependent manner. In case of a radiation and C8PS concurrent treated group, the size of initial tumor was maintained or reduced (FIG. 23). From the aforementioned results, it can be seen that C8PS exhibits the enhancement of radiosensitizing effect both in vitro and in vivo. Meanwhile, tumor growth was suspended at a certain point of time after C8PS alone treatment. It can be seen from this fact that C8PS is useful by itself as an anticancer agent for inhibiting tumor growth.

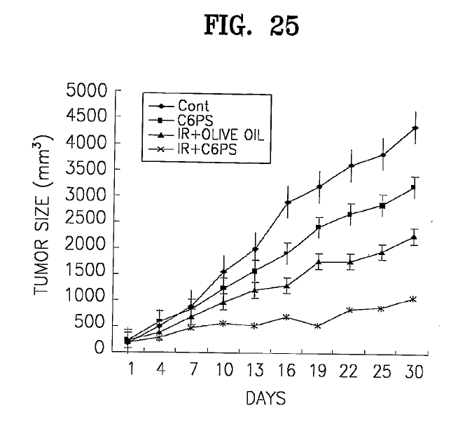

In addition, animal tests demonstrated that C4PS and C6PS induce the enhancement of radiosensitizing effect in vivo. The animal tests were carried out using nude mice of which the femoral regions were transplanted with human uterine cervical cancer cells in the same manner as the aforementioned animal test using C8PS. The results are presented in FIGS. 24 and 25. As shown in FIGS. 24 and 25, a radiation and C4PS (or C6PS) concurrent treated group exhibited significant reduction in tumor size, when compared to a radiation or C4PS (or C6PS) alone treated group. As a result of analysis of correlation between a tumor size and a culture day, the tumor size of a control group rapidly increased in a culture in a day-dependent manner. In the case of a C4PS (or C6PS) alone treated group, the tumor size rapidly increased until 7 days after the treatment. However, after 7 days, the tumor size was slowly increased. The tumor size in a radiation alone treated group slowly increased in a culture in a day-dependent manner. In case of a radiation and C4PS (or C6PS) concurrent treated group, the tumor size showed little changes. From the aforementioned results, it can be seen that C4PS and C6PS exhibit the enhancement of radiosensitizing effect both in vitro and in vivo.

### Acute Oral Toxicity Test of Phytosphingosine and Derivatives Thereof.

Royal Gist-Brocades N.V. (Netherlands) was asked to perform tests for acute oral toxicity and dermal irritation, and Ames tests of phytosphingosine and derivatives thereof. The test results are as follows.

In the acute oral toxicity test, the LD₅₀ (the concentration which induces 50% of cell death) value amounted to 2,000 mg/kg or more in rats. Therefore, it was demonstrated that phytosphingosine and derivatives thereof have excellent physiological safety.

In the dermal irritation test, phytosphingosine and derivatives thereof did not cause dermal damages in rabbits. In addition, the Ames test proved that phytosphingosine and derivatives thereof do not cause mutation.

As apparent from the above description, phytosphingosine and derivatives thereof are useful by themselves for inhibiting various cancers such as human lung cancer, breast cancer, uterine cervical cancer, and blood cancer. At the same time, when phytosphingosine or a derivative thereof is used in combination with radiotherapy, a lowered dose of radiation can be used. Therefore, a relatively high dose radiotherapy effect can be accomplished. For this reason, side effects such as damages to normal cells caused by high dose radiation can be substantially reduced. Therefore, radiotherapy efficiency can be increased.

What is claimed is:

1. A method for treating a cancer comprising:
administering to a human in need of treatment a therapeutically effective amount of a compound represented by formula 1 or a pharmaceutically acceptable salt thereof, wherein the cancer is selected from a group consisting of lung cancer, uterine cancer, breast cancer and blood cell cancer: 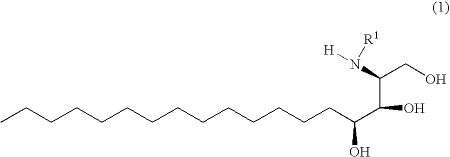
wherein, R¹ is hydrogen or a substituted or unsubstituted C₁-C₂₀ alkylcarbonyl group.

2. The method according to claim 1, wherein R¹ is hydrogen, ethanoyl group, propanoyl group, butanoyl group, pentanoyl group, hexanoyl group, heptanoyl group, octanoyl group, nonanoyl group, decanoyl group, undecanoyl group, or dodecanoyl group.

3. The method according to claim 1, wherein R¹ is hydrogen, butanoyl group, hexanoyl group, or octanoyl group.

4. A method for enhancing radiosensitizing effect comprising:
administering to a human in need of treatment a therapeutically effective amount of a compound represented by formula 1 or a pharmaceutically acceptable salt: 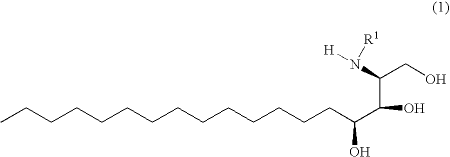
wherein, R¹ is hydrogen or a substituted or unsubstituted C₁-C₂₀ alkylcarbonyl group.

5. The method according to claim 4, wherein R¹ is hydrogen, ethanoyl group, propanoyl group, butanoyl group, pentanoyl group, hexanoyl group, heptanoyl group, octanoyl group, nonanoyl group, decanoyl group, undecanoyl group, or dodecanoyl group.

6. The method according to claim 4, wherein R¹ is hydrogen, butanoyl group, hexanoyl group, or octanoyl group.
